(19) United States
(12) Patent Application Publication
(10) Pub. No.: US 2004/0157318 A1
(43) Pub. Date: Aug. 12, 2004

(54) Device for automatically dispensing microscopic amounts of liquid

(76) Inventors: Henrik Kuhn, Krailling (DE); Michael Mayer, Munich (DE)

Correspondence Address: Ronald R Santucci, Frommer Lawrence & Haug, 745 Fifth Avenue, New York, NY, 10151, US

(21) Appl. No.: 10/473,991

(22) PCT Filed: Mar. 27, 2002
(86) PCT No.: PCT/EP02/03457

(30) Foreign Application Priority Data
Apr. 5, 2001 (DE) 101 17 064.5

(51) Int. Cl. C12M 1/34

(52) U.S. Cl. 435/287.1

(57) ABSTRACT

The present invention relates to a device for automatically dispensing microscopic volumes of fluids comprising at least one movable dispensing head having a plurality of cartridges mounted thereon, a movable object support having at least one object located thereon for receiving the dispensed volumes of fluid, and a control device for controlling the dispensing operation, the cartridges each having a fluid-filled tank, a microdispensing element fluidically connected to the tank, and a mounting element for mounting on the dispensing head.

The present invention relates to a device for automatically dispensing microscopic volumes of fluids, and the uses thereof.

In the laboratory and in production, research and development there is a need for increasingly faster and more precise devices for dispensing fluid in microscopic volume ranges (1-1000 nl) in order to obtain results from the greatest possible amount of data using the smallest possible amounts of chemicals. Especially in the field of combinatorial chemistry, a large number of reactions should take place in parallel in extremely small volumes, it being necessary for the reaction solutions in question to be introduced into the reaction vessels, for example microtitre plates, in microscopic amounts.

Devices for automatically dispensing microscopic volumes of fluids are known. In principle, such devices can be divided into dispensing methods having contact with the surface and contactless dispensing methods.

In the case of dispensing methods having contact with the surface, the surface of the target object is contacted by needles, hollow cannulas, plastics points and the like, the fluid being delivered only on contact. In such methods the surface tension and viscosity of the fluids being dispensed, the adhesion of the fluid to the surface as well as gravitational effects play an important role.

In contrast, in the case of contactless dispensing methods the mentioned components do not make contact with the surface of the target object. Since in such methods the fluid does not adhere to the surface, the surface tension of the fluid has to be overcome solely by the kinetic energy of the fluid as it emerges from the outlet opening. When volumes of less than about 2 μl are to be dispensed, this can take place only if the fluid has very high kinetic energy, which is frequently generated piezoelectrically. For this purpose, there is often used a cylindrical stack of piezoceramics with a glass capillary located therein, the stack being actuated electrically with a voltage pulse. The electric field built up in the piezoceramic results in the contraction of the piezostack and thus in a pressure pulse onto the glass capillary, which expels the fluid. The drops so delivered have a volume in the pl range, depending upon the chamber volume of the glass capillary; larger amounts can be generated additively. The fluids being dispensed are drawn from a fluid reservoir by the dispensing element, which is fluidically connected to a syringe and to a hose, the hose serving also for storing the fluids.

A particular advantage in the case of contactless dispensing method is that it is also possible to dispense onto sensitive materials, because there is no contact with the surface. Furthermore, transfer, that is to say cross-contamination, between the fluids being dispensed can be avoided.

A number of companies currently offer contactless automatic dispensing devices. For example, TECAN AG, which has its headquarters in Hombrechtikon, Switzerland, have recently been marketing an automatic dispensing system under the trade name GENESIS NPS, which is based on a contactless dispensing method. That dispensing system is provided with from 1 to 8 pipetting devices in which the volumes are expelled by means of a piezoceramic. Using special nanopipetting devices it is possible to dispense volumes in the range of from 10 to 700 nl, but only about from 0.1 to 2 nl per shot; if conventional hollow needles are attached, up to 5 ml can be dispensed. Each of the up to 8 pipetting devices can be actuated through its own channel. The dispensing accuracy is less than 5% for an individual channel and less than 10% for a plurality of channels. The fluid being dispensed can be aspirated from reservoir vessels and from 96 well microtitre plates and 384 well microtitre plates; the aspirated fluid can be dispensed into 96 well microtitre plates and 384 well microtitre plates. The dispensing head is moved relative to the support holding the microtitre plates, it being possible for the individual syringes additionally to be lowered in the direction perpendicular to the plane of the microtitre plates.

The TECAN AG pipetting system described by way of example, like all other dispensing systems of other companies known in the prior art, leaves much to be desired in respect of practical use.

A particular disadvantage of such systems is that the fluid for each dispensing operation has to be aspirated into the small number of syringes (inter alia maximum 8) and hoses with dispensing elements, so that each time the fluid is changed the syringes together with the associated hoses and dispensing elements have to be washed clean in order to avoid cross-contamination of the successively aspirated fluids. The washing requires a great deal of time, however, because the washing liquid can be pumped through those elements only at a limited pressure and flow rate. In addition, after washing, the next fluid has to be aspirated again before it can be dispensed, so that time is lost not only in washing but also in aspiration. Fast, freely selectable access to different fluids is thus impeded.

Furthermore, each new dispensing operation is always associated with a dead volume of undispensed fluid which is discarded when the syringes, hoses and dispensing elements are washed. In the case of expensive fluids, such losses can give rise to considerable expense and can even render test series using relatively small expelled amounts of fluid impossible on account of the relatively great losses of fluid that occur. In any case the conventional dispensing systems are not optimum from the economic standpoint.

A disadvantage of conventional automatic dispensing systems is that generally only drops of very small volumes in the pl range can be dispensed by the contactless method. In order to dispense, larger amounts of fluid, especially in the nl and sub-μl range, those dispensing systems therefore need to dispense very rapidly and with a high repetition frequency (up to about 10 kHz). In addition, such dispensing devices readily become blocked, and calibration data must be established for each fluid prior to dispensing, so that fluid-independent dispensing is not possible.

A further disadvantage of most systems known in the prior art arises because dispensing can be carried out only onto objects of a defined format. For example, using the TECAN AG automatic dispensing system, dispensing can be carried out only onto 96 well microtitre plates or 384 well microtitre plates. A dispensing operation onto 1536 well microtitre plates appears doubtful using that system on account of the mechanical problems that arise. It is not possible to dispense onto any desired locations on an object.

It is therefore desirable to have an automatic dispensing system which overcomes the above-mentioned disadvantages of the automatic dispensing systems known in the prior art. Such an automatic dispensing system should especially have substantially more than a maximum of 8 independent dispensing units for dispensing fluids, so that a battery of commonly used fluids is always available. Each of those dispensing units should be capable of being actuated independently of the other dispensing units and capable of dispensing accurate volumes independently of the fluid. Above all, each dispensing unit should be capable of dispensing onto any location on the object, so that dispensing onto any format-free object is possible. Furthermore, the dispensing units should be configured without syringes and hoses, so that every change of fluid does not require further time-consuming washing and aspiration of fluid. Combined with a large number of independent dispensing units it is thus possible to have quick, freely selectable access to the desired fluids. Each dispensing unit should be able to dispense a minimum volume of 1 nl, but above all larger volumes in the range of from 10 to 1000 nl, especially from 50 to 500 nl, should be dispensable in one shot. Especially in the case of small amounts of dispensed fluid, substantially no dead volumes of undispensed fluid should arise. Furthermore, the dispensing units should be cheaper than conventional systems consisting of syringes, hoses and dispensing elements and should be capable of being mounted on the dispensing head so that they can easily and quickly be exchanged. Finally, the object and the dispensing head should be capable of rapid movement relative to one another.

Those aims are achieved by a device for automatically dispensing microscopic volumes of fluids in accordance with claim 1 of the present invention. Advantageous variants of the invention are given by the features of the dependent claims.

The invention provides a device for automatically dispensing microscopic volumes of fluids, the device having at least one movable or immovable dispensing head having a plurality of cartridges mounted thereon, a movable object support having at least one object located thereon for receiving the dispensed volumes of fluid, and a control device for controlling the dispensing operation, and the cartridges each having a fluid-filled tank, a microdispensing element fluidically connected to the tank, and a mounting element for mounting on the dispensing head.

The dispensing device according to the invention, instead of the syringes, hoses and dispensing elements used in conventional dispensing devices, employs a plurality of fluid-filled cartridges which are each fluidically connected to a microdispensing element, each cartridge comprising a tank, a microdispensing element, and a mounting element for mounting on the dispensing head. If the microdispensing elements are integrated into the cartridges, the cartridges act as a protecting shield for the microdispensing devices, which are usually mechanically sensitive, so that practically manageable dispensing units are obtained. The device according to the invention can have one or more dispensing heads, on each of which cartridges are mountable.

Each dispensing head is movable or unmovable; the object support is movable in any case. On the object support there is located at least one object onto which the fluids are to be dispensed. A control device so controls each dispensing operation that one or more cartridges as desired dispense the fluids in their tank onto at least one object. Fluids that come into consideration are, for example, solutions of substances in solvents, such as, for example, water, methanol, dimethylformamide (DMF), dimethyl sulfoxide (DMSO), dichloromethane, benzene and tetrahydrofuran (THF), and also suspensions.

Microdispensing elements suitable for use according to the invention are known. In this connection reference is made to the two patent specifications DE 198 02 367 C1 and DE 198 02 368 C1 of Hahn-Schickard-Gesellschaft für angewandle Forschung e.V., Stuttgart, D E. Such microdispensing elements comprise a dispensing chamber having a deflectable diaphragm, which chamber can be filled with fluid, an actuating device for deflecting the diaphragm, and an outlet nozzle. The dispensing chamber is filled with fluid before the operation of expelling fluid begins and is then actuated by the actuating device, usually stacked piezoceramics, so that the diaphragm is deflected into the interior of the dispensing chamber and the fluid is expelled through the outlet nozzle. After the end of the expelling operation, the dispensing chamber is filled with fluid again as the diaphragm returns to its starting position and is ready for the next expelling operation.

In the device according to the invention it is preferable for the object support to be movable in the horizontal (x, y) plane and optionally also in the vertical z-direction. The dispensing head(s) can in that case be movable or immovable. When one or more dispensing heads are immovable, relative movement between the dispensing head and the object support is effected solely by movement of the object support, which then takes place preferably in the (x, y) plane and optionally also in the z-direction. Movement of the object support in the z-direction is unnecessary especially when the objects used each have the same dimensions in the z-direction, so that a respective adjustment of the relative distance between the object support and the dispensing head in the z-direction is unnecessary.

When a dispensing head is movable, it is preferable for the dispensing head to be movable in the horizontal (x, y) plane and optionally additionally along the vertical z-direction or only along the vertical z-direction. Movement of a dispensing head in the z-direction is unnecessary especially when the objects used each have the same dimensions in the z-direction, so that a respective adjustment of the relative distance between the object support and the dispensing head in the z-direction is unnecessary.

When only one movable dispensing head is present it is advantageous for the dispensing head to be movable solely along the vertical z-direction. In that case the object support performs a movement solely in the (x, y) plane, so that the object support and the dispensing head are moved closer to one another or further away from one another by movement of the dispensing head in the vertical z-direction.

Equally it is of advantage if, in the event of movement of the object support in the vertical z-direction, a dispensing head is moved only in the (x, y) plane, so that the object support and the dispensing head are moved closer to one another or further away from one another solely by movement of the object support in the vertical z-direction.

Movement of a dispensing head in the (x, y) plane also includes one-dimensional movement of the dispensing head in the x-direction or the y-direction.

In a special embodiment of the invention, at least one dispensing head is mounted on at least one dispensing head support. This implies that one or more dispensing heads can be mounted on a dispensing head support. Equally possible is an arrangement of one or more dispensing head supports. A dispensing head support is arranged to be movable parallel to the x- or y-direction of the horizontal (x, y) plane. Each dispensing head is mounted to be movable on the dispensing head support and can be moved along the dispensing head support, that is to say either in the x-direction or the y-direction, according to the arrangement of the dispensing head support. Movement in the horizontal (x, y) plane of a dispensing head is thus obtained by a combination of a one-dimensional movement of the dispensing head along the dispensing head support and a one-dimensional movement of the dispensing head support in the direction perpendicular thereto.

In an especially advantageous and preferred embodiment of the invention, the dispensing head(s) are moved solely in order to assume a working position in which dispensing is to occur, or to assume a rest position in which dispensing is not intended. This also includes the case where a dispensing head is moved from one working position into another working position. Once a dispensing head has assumed a working position, that is to say by movement of the dispensing head in the horizontal (x, y) plane and in the vertical z-direction perpendicular thereto, or by movement only in the horizontal (x, y) plane, or by movement only in the vertical z-direction, the dispensing head remains static during the dispensing operation and solely movement of the object support in the horizontal (x, y) plane takes place. The movement of the dispensing head in the vertical z-direction can be supplemented or replaced by movement of the object support in the vertical z-direction. In the latter case, the dispensing head is moved only in the (x, y) plane.

When the dispensing operation is complete, a dispensing head can be brought by fresh movement in the horizontal (x, y) plane and in the vertical z-direction perpendicular thereto, or only in the (x, y) horizontal plane, or only in the vertical z-direction, into a working position in which it remains static for the subsequent dispensing operation and solely movement of the object support in the horizontal (x, y) plane takes place. The movement of the dispensing head in the vertical z-direction can again be supplemented or replaced by movement of the object support in the vertical z-direction. In the latter case, the dispensing head is moved only in the (x, y) plane.

A dispensing head can be brought by movement in the horizontal (x, y) plane and in the vertical z-direction perpendicular thereto, or only in the horizontal (x, y) plane, or only in the vertical z-direction, from a working position into a rest position, it being possible for the movement of the dispensing head in the vertical z-direction to be supplemented or replaced by movement of the object support in the vertical z-direction. In the latter case, the dispensing head is moved only in the (x, y) plane.

When an immovable dispensing head is present, the working position and the rest position are assumed by the object support. When the object support is in a working position, dispensing of fluids can take place, whereas when the object support is in a rest position such dispensing of fluids is not intended. In a rest position of the object support, the object support is preferably spaced apart from the dispensing head, so that the dispensing head and especially the cartridges are freely accessible.

The speeds and accelerations of a dispensing head are considerably lower in the case of such movements than the speeds and accelerations imposed on the object support during automatic dispensing.

Unlike the comparatively slow relative movements and accelerations between object support and dispensing head employed in the case of conventional systems, the object support here is preferably movable at a speed of at least 800 mm/s and an acceleration of at least 10 m/s². This can be achieved, for example, by driving the object support by means of wound linear drives or by means of a two-dimensional stepping motor, in each case having accurate positional resolution. According to the invention, bidirectional positional resolution on the object of less than 50 μm is preferred.

In an especially advantageous embodiment of the invention, the cartridges can be mounted manually and/or automatically, individually and/or in blocks on a dispensing head by means of their mounting element(s). The cartridges can, for example, be mounted on the dispensing head by means of a clip connection. This enables the dispensing head to be fitted with cartridges quickly without complicated fitting and/or servicing work. It is also readily possible to exchange cartridges when, for example, the fluids in those cartridges are no longer required or the cartridges are empty. Automatic fitting of cartridges to the dispensing heads can be effected, for example, by mounting the cartridges on the respective dispensing head by means of a robot gripper arm. It is equally possible for a robot gripper arm or an operator to place the cartridges onto the object support and for the dispensing head in question to be moved over the cartridges and then lowered onto the cartridges so that the cartridges become mounted on the dispensing head.

It is also preferable for a dispensing head to have an automatic cartridge ejection device so that the cartridges mounted on the dispensing head can automatically be ejected individually and/or in blocks. Alternatively it is possible for the cartridges to be removed, for example, by a robot arm or by an operator. Fitting, removing or exchanging the cartridges is preferably carried out in a rest position of the dispensing head, that is to say in a position in which no dispensing is intended, which position is preferably located to one side of the object. Where there is a plurality of dispensing heads, the fitting, removing or exchanging of the cartridges or any servicing work to the dispensing head can always be carried out when at least one other dispensing head is in an operating position ready for dispensing, so that, for example, virtually no time is lost when replacing/exchanging fluids. When a dispensing head is immovable, the fitting, replacing or exchanging of the cartridges on the dispensing head is preferably effected in a rest position of the object support.

In the device according to the invention it is especially advantageous when the cartridges and the microdispensing elements, that is to say all the elements coming into contact with fluid, are chemically resistant to aggressive substances. For example, the cartridges can be manufactured from plastics, such as, for example, polypropylene (PP), Peek, Teflon and the like. The microdispensing elements can be produced, for example, from silicon, plastics, such as, for example, PP, Peek, Teflon and the like, and Pyrex.

It is also possible for the cartridges to be used for the long-term storage of the fluids. For example, it is possible to insert fluid-filled cartridges from an external store into the device according to the invention, without it being necessary to transfer the fluid from a reservoir vessel to the cartridges with the risk of possible contamination of the fluid and/or contamination of the environment/operating personnel. Accordingly, the fluid-filled cartridges can also be left in the device according to the invention for a prolonged period.

It is also preferred according to the invention that the cartridges be refillable with fluid. Expense can advantageously be spared by using cartridges a number of times.

In a preferred variant of the invention the cartridges each have a volume of from 1 to 5 ml, advantageously 2 ml.

The cartridges mounted on the dispensing head of the device according to the invention may contain the same or different fluids. The cartridges can especially all be filled with different fluids, so that a battery of all commonly used substances is always kept in readiness. Unlike conventional automatic dispensing devices, therefore, it is unnecessary for a dispensing unit or cartridge to be used for different fluids. Each fluid has its “own” cartridge. It is likewise unnecessary to wash the cartridges, so that practically no dead-volume of undispensed fluid is lost. Furthermore, expensive substances can be used in multiple test series.

It is also advantageous for the cartridges each to be provided with an electrically readable code. Such a code can be stored, for example, using an EEPROM, HF tag, barcode or the like. The code can contain fluid- or cartridge-specific data, such as, for example, substance identification, solvent identification, filling date, ageing, purity and the like. Such data can be supplied directly to the test planning means, so that optimum use or availability of substances is always ensured. When a cartridge is changed, by virtue of the cartridge-specific data it is accordingly unnecessary for the operator to indicate the position to be fitted, because the system is able to recognise it automatically.

The cartridges are preferably arranged on the dispensing head in the form of a matrix. A 10×10 matrix having a total of 100 cartridges and a 8×12 matrix having a total of 96 cartridges have proved advantageous. If the matrix-like arrangement is such that each cartridge covers a base unit of a grid 9×18 mm in size, compatibility with 96, 384 and 1536 well microtitre plates, at least in one dimension, can advantageously be established. When a cartridge covers a base unit of a grid 9×9 mm in size, registration with 96 well microtitre plates can advantageously be achieved.

The control unit is preferably so configured that each cartridge can be actuated separately independently of the other cartridges. It is equally possible for some or all of the cartridges to be actuated together. For example, each individual cartridge and each freely selectable location on an object can be brought into a position one above the other and a selectable amount of fluid expelled. This advantageously makes it possible to dispense onto a format-free object. In particular, it is not necessary to use microtitre plates as target objects. Instead it is possible to dispense onto any desired object, for example synthesis plates, with a high degree of positional resolution.

In an especially advantageous embodiment of the device according to the invention the cartridges are each suitable for dispensing volumes in the range of from 1 to 1000 nl, preferably from 10 to 1000 nl and especially from 50 to 500 nl, so that unlike conventional devices substantially greater volumes can be dispensed per single shot and the losses of fluid through evaporation can be minimised as result

Furthermore, the cartridges are each suitable for dispensing at a volume rate of >100 nl/s, preferably >500 nl/s. Moreover, using the cartridges it is especially possible to carry out dispensing with an error of less than 5% by volume over all fluids, a cone scatter of a maximum of ±5° occurring during dispensing.

The device according to the invention can have a heating/cooling device and in addition a thermally insulating cover and/or one or more intermediate walls. It is thus possible for the cartridges and/or the target object to be cooled or heated as desired. For example, the temperature for storing the fluids can be suitably selected or the consistency of certain fluids can be suitably adjusted for the dispensing operation. Furthermore, the temperature of one or more objects can be adjusted so as to favour the course of certain reactions. In particular, different objects can be maintained at different temperatures.

The device according to the invention is advantageously used for dispensing fluids in chemical synthesis in fluid- and solid-phase systems, especially in the field of combinatorial chemistry where the ability to dispense small volumes of often expensive substances is crucial. In addition, it must be possible to have freely selectable fast access to a certain battery of substances being used. For this purpose there are especially advantageously used dispensing heads which are fitted with at least 200 cartridges.

The device according to the invention can also be used for biological and diagnostic screening. For this purpose there are especially advantageously used dispensing heads which are fitted with at least 10 cartridges.

A special embodiment of the present invention is described below with reference to the accompanying drawings.

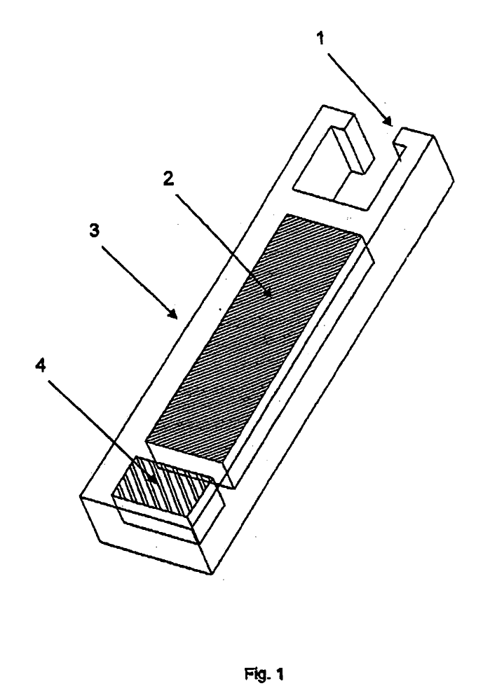

FIG. 1 is a perspective view of a cartridge of the dispensing device according to the invention:

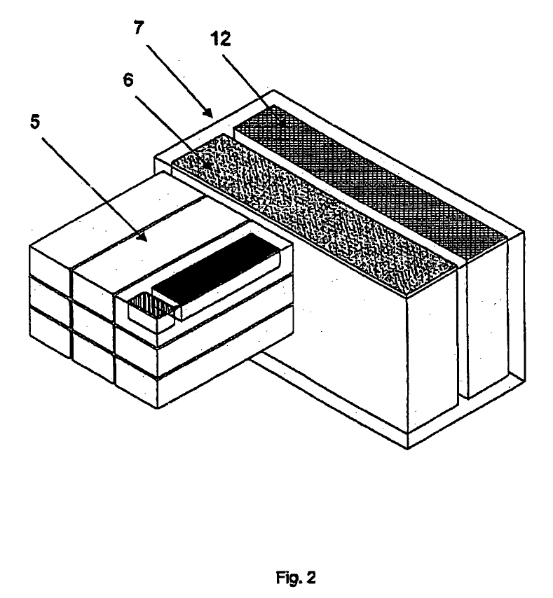

FIG. 2 is a perspective view of a dispensing head of the dispensing device according to the invention;

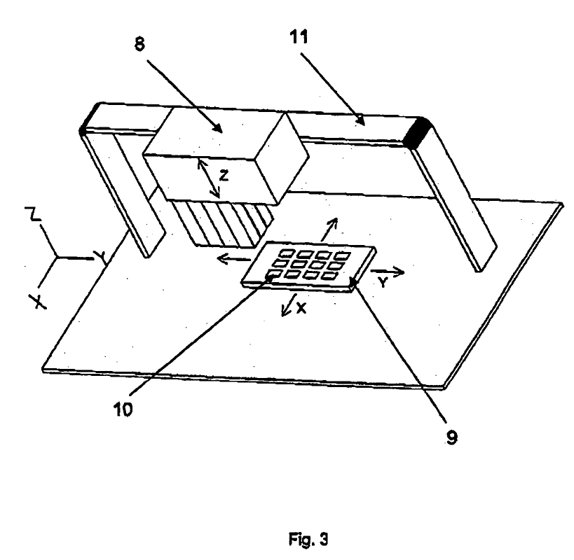

FIG. 3 is a perspective view in diagrammatic form of the structure of a dispensing device according to the invention having a dispensing head on a dispensing head support; and

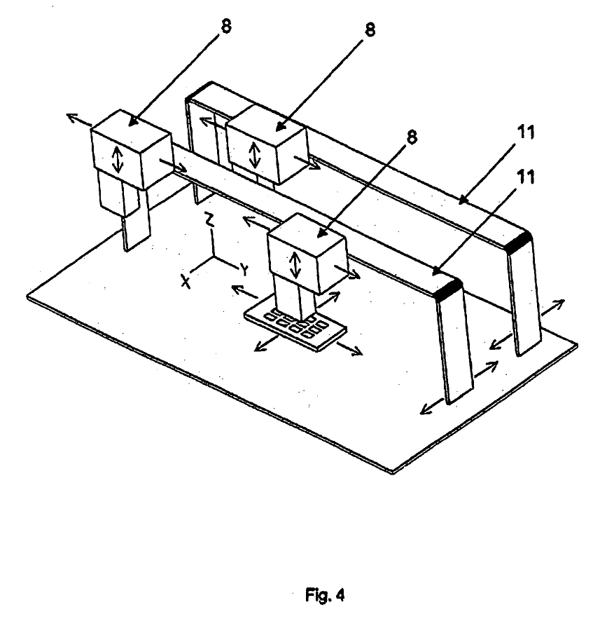

FIG. 4 shows a perspective view in diagrammatic form of the structure of a dispensing device according to the invention having three dispensing heads on two dispensing head supports.

As can be seen from FIG. 1, a cartridge 3 of the dispensing device according to the invention has a tank 2, a microdispensing element 4 and a clip connection 1. By means of the clip connection 1, the cartridge 3 can be mounted on the clip mechanism 6 of the mounting head 7, and especially quickly fitted, removed or exchanged without complicated fitting/servicing work. By virtue of the compact structure of the cartridge 3 with an integrated arrangement of the microdispensing element 4, the microdispensing element 4, which is usually very mechanically sensitive, is protected by the cartridge housing.

FIG. 2 shows a dispensing head 8 of the dispensing device according to the invention. The dispensing head 8 comprises a mounting head 7 with a matrix-like arrangement 5 of cartridges 3 mounted thereon. The mounting head 7 is provided with a clip mechanism 6 for clipping on the cartridges 3, and with an automatic cartridge ejection device 6 for automatically ejecting the cartridges. Also located therein is an electronic element with which the automatic ejection of the cartridges 3 is controlled. The interface electronics for the individual cartridges 3 is located in 12.

FIG. 3 shows in diagrammatic form the structure of a dispensing device according to the invention having a dispensing head 8 on a dispensing head support 11. As can be seen from FIG. 3, the dispensing head 8 is mounted on a U-shaped dispensing head support 11. The dispensing head support 11 is arranged along the x-direction of the horizontal (x, y) plane. In this variant the dispensing head 8 can be moved only in the vertical z-direction. A working or rest position of the dispensing head 8 is accordingly assumed solely by movement of the dispensing head 8 along the z-direction. The working position and the rest position of the dispensing head 8 can be identical. The dispensing head support 11 is immovable. The object 10 onto which dispensing is to be carried out is located on the object support 9. The object support 9 is movable in the horizontal (x, y) plane.

For a dispensing operation, the dispensing head 8 assumes its working position along the z-direction, that is to say it is lowered or raised until it reaches a distance from the object 10 suitable for the dispensing operation. In its working position, the dispensing head 8 remains static for the dispensing operation, that is to say no further movement of the dispensing head 8 along the z-direction takes place. The setting of the relative positions between the cartridges 3 and the object 10 in the (x, y) plane necessary for the dispensing operation is effected solely by movement of the object support in the (x, y) plane. When the dispensing operation is complete, the dispensing head 8 can be brought into a rest position by movement along the z-direction. In particular, for this purpose the object support 8 can be lowered onto the object support.

FIG. 4 shows in diagrammatic form the structure of a dispensing device according to the invention having three dispensing heads 8 on two dispensing head supports 11. The basic structure of the dispensing heads 8 and of the dispensing head supports 11 agrees with that shown in FIG. 3. The dispensing head supports 11 are arranged parallel to the x-direction of the (x, y) plane and are movable in the y-direction. The dispensing heads 3 are movable along the dispensing head supports 11 parallel to the x-direction. Movement of the dispensing heads 8 in the (x, y) plane is therefore effected by a combination of the respective one-dimensional movement of the dispensing heads 8 in the x-direction and of the dispensing head supports 11 in the y-direction. In addition, the dispensing heads 8 are movable in the z-direction. The object support 9 is movable in the (x, y) plane.

Movement of the dispensing heads 8 or the dispensing head supports 11 is effected solely for the purpose of bringing the dispensing heads 8 from a rest position into a working position, from a working position into a different working position, or from a working position into a rest position. Once a dispensing head 8 has been brought into a working position, it remains static during automatic dispensing. The setting of the relative positions between cartridges 3 and object 10 necessary for dispensing is effected solely by movement of the object support 9 in the (x, y) plane. The speeds and accelerations of the dispensing heads 8 and dispensing head supports 11 are therefore far lower than those of the object support 10.

1. Device for automatically dispensing microscopic volumes of fluids, for use in dispensing fluids in chemical synthesis in liquid- and solid-phase systems and in biological and diagnostic screening, comprising at least one movable or immovable dispensing head (8) having a plurality of cartridges (3) mounted thereon, a movable object support (9) having at least one object (10) located thereon for receiving the dispensed volumes of fluid, and a control device for controlling the dispensing operation, the cartridges (3) each having a fluid-filled tank (2), a microdispensing element (4) fluidically connected to the tank (2), and a mounting element (1) for mounting on the dispensing head (8), and the cartridges (3) being individually mountable or exchangeable on a dispersing head (8) manually and/or automatically.

2. Device according to claim 1, characterised in that the object support (9) is movable in the horizontal plane (x, y) and optionally along the vertical direction (z).

3. Device according to claim 1 or 2, characterised in that a dispensing head (8) is movable in the horizontal plane (x, y) and optionally along the vertical direction (z) or only along the vertical direction (z).

4. Device according to claim 3, characterised in that either a dispensing head (8) or the object support is moved In the vertical direction (z).

5. Device according to any one of the preceding claims, characterised in that at least one dispensing head (8) is mounted on at least one dispensing head support (11) arranged parallel to the x- or y-direction of the horizontal plane (x, y) so as to be movable along the dispensing head support (11) and optionally in the z-direction, the dispensing head support (11) being movable perpendicular to the direction of its arrangement in the x- or y-direction and the movement of a dispensing head (8) in the horizontal plane being effected by a combination of a movement of the dispensing head (8) along the dispensing head support (11) and a movement of the dispensing head support (11) in the direction perpendicular thereto.

6. Device according to any one of the preceding claims, characterised in that a dispensing head (8) is moved only in order to assume a working position or a rest position of the dispensing head (8), and in a working position of the dispensing head (8) only movement of the object support (9) takes place.

7. Device according to any one of the preceding claims, characterised in that the object support (9) is movable at a speed of at least 800 mm/s and an acceleration of at least 10 m/s².

8. Device according to any one of the preceding claims, characterised in that the object support (9) is driven in the (x, y) plane by means of a wound linear drive.

9. Device according to any one of claims 1 to 7, characterised in that the object support (9) is driven in the (x, y) plane by means of a two-dimensional stepping motor.

10. Device according to any one of the preceding claims, characterised in that a dispensing head (8) is provided with a cartridge ejection device (6) for automatically ejecting cartridges (3).

11. Device according to any one of the preceding claims, characterised in that the cartridges (3) and the microdispensing elements (4) are chemically resistant.

12. Device according to any one of the preceding claims, characterised in that the cartridges (3) are suitable for storing the fluids.

13. Device according to any one of the preceding claims, characterised in that the cartridges (3) each have a volume of from 1 to 5 ml, especially 2 ml.

14. Device according to any one of the preceding claims, characterised in that the cartridges (3) are each provided with an electrically readable code.

15. Device according to any one of the preceding claims, characterised in that the control unit is suitable for separately controlling the cartridges (3) of each dispensing head (8).
